(19) United States
(12) Patent Application Publication
(10) Pub. No.: US 2021/0127985 A1
(43) Pub. Date: May 6, 2021

(54) Secure Patient Data

(71) Applicant: West Affum Holdings Corp., Grand Cayman (KY)

(72) Inventor: Steven E. Sjoquist, Lynnwood, WA (US)

(73) Assignee: West Affum Holdings Corp., Grand Cayman (KY)

(21) Appl. No.: 17/085,834

(22) Filed: Oct. 30, 2020

Related U.S. Application Data
(60) Provisional application No. 62/929,801, filed on Nov. 2, 2019.

(51) Int. Cl. A61B 5/0205; A61N 1/39; A61B 5/33; A61B 5/333; A61B 5/282; A61B 5/364; A61B 5/11; G16H 10/60; G06F 21/60; H04L 9/08

(52) U.S. Cl. CPC A61B 5/0205; A61N 1/3904; A61B 5/33; A61B 5/333; A61B 5/282; A61B 5/02438; A61B 5/1118; G16H 10/60; G06F 21/602; H04L 9/0894; H04L 9/0819; A61B 5/364

(57) ABSTRACT

In one embodiment, a method to store data collected by a wearable cardioverter defibrillator (WCD) is described. The method includes connecting to at least one sensor and obtaining a signal from the at least one sensor. The method also includes analyzing the signal from the at least one sensor into usable data and cataloguing the data into one or more segments. The method encrypts the one or more segments and sends the encrypted one or more segments to a verified distributed network.

### CROSS REFERENCE TO RELATED PATENT APPLICATIONS

This patent application claims benefit of U.S. Provisional Patent Application No. 62/929,801 filed Nov. 2, 2019 and is incorporated herein by reference in their entirety for all purposes.

### BACKGROUND

When people suffer from some types of heart arrhythmias, in some instances, blood flow to various parts of the body may be reduced. Some arrhythmias can result in a Sudden Cardiac Arrest (SCA). SCA can lead to death very quickly, e.g. within 10 minutes, unless treated in the interim. Some observers have thought that SCA is the same as a heart attack, which it is not.

Some people have an increased risk of SCA. Such people may include patients who have had a heart attack or a prior SCA episode. A frequent recommendation for these people is to receive an Implantable Cardioverter Defibrillator (ICD). The ICD is surgically implanted in the chest, and continuously monitors the patient's intracardiac electrogram (IEGM). If certain types of heart arrhythmias are detected, then the ICD delivers an electric shock through the heart.

As a further precaution, people who have been identified to have an increased risk of a SCA are sometimes given a Wearable Cardioverter Defibrillator (WCD) system to wear until an ICD is implanted. Early versions of such systems were called wearable cardiac defibrillator systems. A WCD system typically includes a harness, vest, belt, or other garment that the patient wears. The WCD system further includes electronic components, such as a defibrillator and electrodes, coupled to the harness, vest, or another garment. When the patient wears the WCD system, the electrodes may electrically contact the patient's skin, and aid in sensing the patient's electrocardiogram (ECG). If a shockable heart arrhythmia (e.g., ventricular fibrillation or VF) is detected from the ECG, then the defibrillator delivers an appropriate electric shock through the patient's body, and thus through the heart. The delivered shock may restart the patient's heart and save the patient's life.

### BRIEF SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure describes instances and examples of cardiac monitoring systems (e.g., WCD systems), devices, systems, storage media that may store programs, data, and methods.

In one embodiment, a method to store data collected by a wearable cardioverter defibrillator (WCD) is described. The method includes connecting to at least one sensor and obtaining a signal from the at least one sensor. The method also includes analyzing the signal from the at least one sensor into usable data and cataloguing the data into one or more segments. The method encrypts the one or more segments and sends the encrypted one or more segments to a verified distributed network.

In some embodiments, the method may store the encryption key and distribute the encryption key to authorized personnel. The method may include receiving a public and private identifier for the verified distributed network, locally storing the public and private identifier, and distributing the public and private identifier to authorized personnel. In some embodiments, sending the encrypted one or more segments may include initiating a new data chain in the verified distributed network. In some embodiments, the method may include initiating segment mining to the remote distributed network.

In some embodiments, the data may include one of heart rate data, defibrillator status data, wearability data, patient movement data, heartrate over time, heartrate histogram, steps over time, detected electrocardiogram arrhythmias, and some combination thereof. In another embodiment, the segments may be organized by at least one of a date, time, duration, cardiac event, or some combination thereof.

In another embodiment, a WCD system for monitoring health of a patient wearing the WCD system is described. The system includes at least one sensor positioned to gather data about the patient and one or more memories. The one or more memories are configured to store patient data. The system also includes one or more processors. The processors are configured to cause the system to receive a signal from the at least one sensor, analyze the signal from the at least one sensor into usable data, catalogue the usable data into one or more segments, encrypt the one or more segments, and send the one or more encrypted segments to a remote distributed network.

In some embodiments, the processor may be further configured to store the encryption key and distribute the encryption key to authorized personnel. In another embodiment, the processor may be further configured to receive a public and private identifier for the verified distributed network, locally store the public and private identifier, and distribute the public and private identifier to authorized personnel. In another embodiment, sending the encrypted one or more segments may include initiating a new data chain in the verified distributed network.

In some embodiments, the processor may be further configured to initiate segment mining to the remote distributed network. In some embodiments, the data may include one of heart rate data, defibrillator status data, wearability data, patient movement data, heartrate over time, heartrate histogram, steps over time, detected electrocardiogram arrhythmias, and some combination thereof. In some embodiments, the segments may be catalogued by at least one of a date, time, duration, cardiac event, or some combination thereof.

In another embodiment, a method to store patient data in a remote distributed network is described. The method includes positioning at least one electrocardiogram (ECG) sensing electrodes to measure electrical activity of a heart of a person and receiving at least one ECG signal from the at least one ECG electrode. The method also includes analyzing the signal into usable data including at least one of a heartrate over time, heartrate histogram, and detected ECG arrhythmias. The method further includes cataloguing the usable data into one or more segments based at least in part on a size of the data and encrypting the one or more segments. The method also includes sending the one or more encrypted segments to a remote distributed network.

### BRIEF DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a diagram of a sample WCD system in accordance with exemplary embodiments described herein;

FIG. 2 is a block diagram of an example defibrillator in accordance with exemplary embodiments described herein;

FIG. 3 is a diagram of sample embodiments of components of a WCD system in accordance with exemplary embodiments described herein;

FIG. 4 is a block diagram of an example defibrillator in accordance with exemplary embodiments described herein;

FIG. 5 is a is a block diagram of an exemplary distributed network storage system in accordance with exemplary embodiments described herein; and

FIG. 6 is exemplary flow diagram in accordance with exemplary embodiments described herein.

### DETAILED DESCRIPTION

The detailed description set forth below in connection with the appended drawings, where like numerals reference like elements, are intended as a description of various embodiments of the present disclosure and are not intended to represent the only embodiments. Each embodiment described in this disclosure is provided merely as an example or illustration and should not be construed as precluding other embodiments. The illustrative examples provided herein are not intended to be exhaustive or to limit the disclosure to the precise forms disclosed.

In the following description, specific details are set forth to provide a thorough understanding of exemplary embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure. Further, it will be appreciated that embodiments of the present disclosure may employ any combination of features described herein.

Wearable Cardioverter Defibrillators (WCDs) are worn by patients at risk for sudden cardiac arrest. When a patient wears a WCD, the WCD may store patient data. For example, the WCD may send patient medical data to one or more online systems. In some instances, the medical data may be stored completely within a centralized data system. The medical data may be accessed or viewed via proprietary applications. The centralized storage may limit access to other external systems rendering some data inaccessible. If a system needs to access patient data, the data must be transferred to the other system which may involve transforming the data to a format for the other system to import. The single system data storage complicates aggregating data from multiple sources and once data is transferred, it may be duplicated and subject to change which may not be easily tracked or recorded.

In an embodiment, the WCD system disclosed may use blockchain technology for patient data storage. For example, the system may encode patient data into blocks which may be sent to a blockchain system on the Internet. In some embodiments, small amounts of data may be encrypted and stored in small amounts called blocks. The blocks for a single patient may link together forming a chain.

Once stored in the block and connected to the chain, the blockchain technology may store the blocks securely and immutably. For instance, the blockchain technology may grant access to the data but the data itself cannot be changed. If any changes are made to the data, the changes may be saved in new blocks and added to the chain. This provides a record of changes to any data stored. Additionally, the blockchain technology may provide access mechanisms of various types. The blocks in the chain become a ledger of incrementally generated data for the given patient during their prescription cycle. When the prescription is completed, the blockchain contains the complete set of medical records of that patient as provided by the heart monitor. The data includes heart rate over time, heart rate histogram, steps over time, and episodes that patient experiences such as for detected ECG arrhythmias.

Various types of data systems can access the blocks in the blockchain to gain access to the data stored therein. This allows different systems to access multiple patient databases securely and remotely without needing to convert the data into different formats. Some data systems that may access the patient data include, but are not limited to, remote patient monitoring, electronic medical records, business uses, medical data aggregation, and the like.

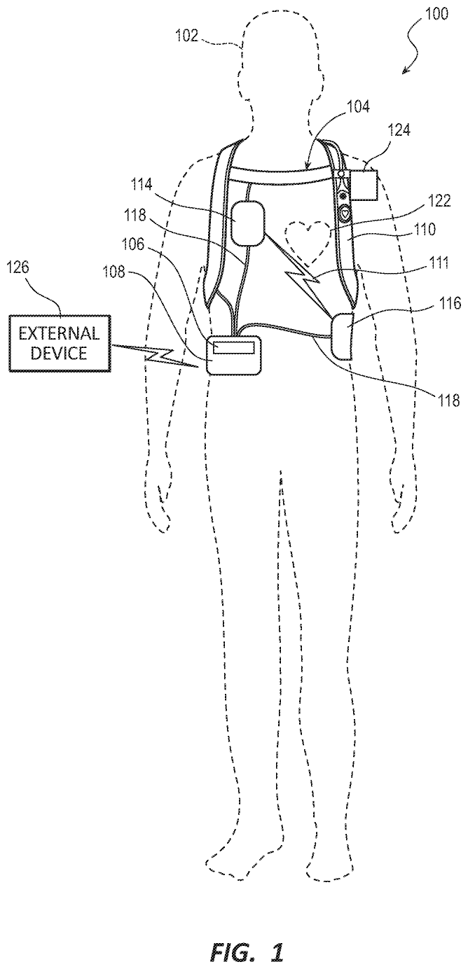

FIG. 1 illustrates a system 100 with a patient 102 wearing an example of a WCD system 104 according to embodiments described herein. In some embodiments, the WCD system 104 may include one or more communication devices 106, a support structure 110, and an external defibrillator 108 connected to two or more defibrillation electrodes 114, 116, among other components.

The support structure 110 may be worn by the patient 102. The patient 102 may be ambulatory, meaning the patient 102 can walk around and is not necessarily bed-ridden while wearing the wearable portion of the WCD system 104. While the patient 102 may be considered a “user” of the WCD system 104, this is not a requirement. For instance, a user of the WCD system 104 may also be a clinician such as a doctor, nurse, emergency medical technician (EMT) or other similarly tasked individual or group of individuals. In some cases, a user may even be a bystander. The particular context of these and other related terms within this description should be interpreted accordingly.

In some embodiments, the support structure 110 may include a vest, shirt, series of straps, or other system enabling the patient 102 to carry at least a portion of the WCD system 104 on the patient's body. In some embodiments, the support structure 110 may comprise a single component. For example, the support structure 110 may comprise a vest or shirt that properly locates the WCD system 104 on a torso 112 of the patient 102. The single component of the support structure 110 may additionally carry or couple to all of the various components of the WCD system 104.

In other embodiments, the support structure 110 may comprise multiple components. For example, the support structure 110 may include a first component resting on a patient's shoulders. The first component may properly locate a series of defibrillation electrodes 114, 116 on the torso 112 of the patient 102. A second component may rest more towards a patient's hips, whereby the second component may be positioned such that the patient's hips support the heavier components of the WCD system 104. In some embodiments, the heavier components of the WCD system 104 may be carried via a shoulder strap or may be kept close to the patient 102 such as in a cart, bag, stroller, wheelchair, or other vehicle.

The external defibrillator 108 may be coupled to the support structure 110 or may be carried remotely from the patient 102. The external defibrillator 108 may be triggered to deliver an electric shock to the patient 102 when patient 102 wears the WCD system 104. For example, if certain thresholds are exceeded or met, the external defibrillator 108 may engage and deliver a shock to the patient 102.

The defibrillation electrodes 114, 116 can be configured to be worn by patient 102 in a number of ways. For instance, the defibrillator 108 and the defibrillation electrodes 114, 116 can be coupled to the support structure 110 directly or indirectly. For example, the support structure 110 can be configured to be worn by the patient 102 to maintain at least one of the electrodes 114, 116 on the body of the patient 102, while the patient 102 is moving around, etc. The electrodes 114, 116 can be thus maintained on the torso 112 by being attached to the skin of patient 102, simply pressed against the skin directly or through garments, etc. In some embodiments, the electrodes 114, 116 are not necessarily pressed against the skin but becomes biased that way upon sensing a condition that could merit intervention by the WCD system 104. In addition, many of the components of defibrillator 108 can be considered coupled to support structure 110 directly, or indirectly via at least one of defibrillation electrodes 114, 116.

The WCD system 104 may defibrillate the patient 102 by delivering an electrical charge, pulse, or shock 111 to the patient 102 through a series of electrodes 114, 116 positioned on the torso 112. For example, when defibrillation electrodes 114, 116 are in good electrical contact with the torso 112 of patient 102, the defibrillator 108 can administer, via electrodes 114, 116, a brief, strong electric pulse 111 through the body. The pulse 111 is also known as shock, defibrillation shock, therapy, electrotherapy, therapy shock, etc. The pulse 111 is intended to go through and restart heart 122, in an effort to save the life of patient 102. The pulse 111 can further include one or more pacing pulses of lesser magnitude to pace heart 122 if needed. The electrodes 114, 116 may be electrically coupled to the external defibrillator 108 via a series of electrode leads 118. The defibrillator 108 may administer an electric shock 111 to the body of the patient 102 when the defibrillation electrodes 114, 116 are in good electrical contact with the torso 112 of patient 102. In some embodiments, devices (not shown) proximate the electrodes 114, 116 may emit a conductive fluid to encourage electrical contact between the patient 102 and the electrodes 114, 116.

In some embodiments, the WCD system 104 may also include either an external or internal monitoring device or some combination thereof. FIG. 1 displays an external monitoring device 124 which may also be known as an outside monitoring device. The monitoring device 124 may monitor at least one local parameter. Local parameters may include a physical state of the patient 102 such as ECG, movement, heartrate, pulse, temperature, and the like. Local parameters may also include a parameter of the WCD 104, environmental parameters, or the like. The monitoring device 124 may be physically coupled to the support structure 110 or may be proximate the support structure 110. In either location, the monitoring device 124 is communicatively coupled with other components of the WCD 104.

For some of these parameters, the device 124 may include one or more sensors or transducers. Each one of such sensors can be configured to sense a parameter of the patient 102, and to render an input responsive to the sensed parameter. In some embodiments, the input is quantitative, such as values of a sensed parameter; in other embodiments, the input is qualitative, such as informing whether or not a threshold is crossed. In some instances, these inputs about the patient 102 are also referred to herein as patient physiological inputs and patient inputs. In some embodiments, a sensor can be construed more broadly, as encompassing many individual sensors.

In some embodiments, a communication device 106 may enable the patient 102 to interact with, and garnish data from, the WCD system 104. The communication device 106 may enable a patient or third party to view patient data, dismiss a shock if the patient is still conscious, turn off an alarm, and otherwise engage with the WCD system 104. In some instances, the communication device 106 may transfer or transmit information include patient data to a third-party data server such as a cloud server or a blockchain server. In some embodiments, the communication device 106 may be a separable part of an external defibrillator 108. For example, the communication device 106 may be a separate device coupled to the external defibrillator 108. In some embodiments, the communication device 106 may be wired or wirelessly linked to the external defibrillator 108 and may be removable from the defibrillator 108. In other embodiments, the communication device 106 may form an inseparable assembly and share internal components with the external defibrillator 108. In some embodiments, the WCD system 104 may include more than one communication device 106. For example, the defibrillator 108 may include components able to communicate to the patient and the WCD system 104 may include a separate communication device 106 remote form the defibrillator 108.

In some embodiments, the defibrillator 108 may connect with one or more external devices 126. For example, as shown in FIG. 1, the defibrillator 108 may connect to various external devices 126 such as a the cloud, a remote desktop, a laptop, a mobile device, or other external device using a network such as the Internet, local area networks, wide area networks, virtual private networks (VPN), other communication networks or channels, or any combination thereof.

In embodiments, one or more of the components of the exemplary WCD system 104 may be customized for the patient 102. Customization may include a number of aspects including, but not limited to, fitting the support structure 110 to the torso 112 of patient 102; baseline physiological parameters of patient 102 can be measured, such as the heart rate of patient 102 while resting, while walking, motion detector outputs while walking, etc. The measured values of such baseline physiological parameters can be used to customize the WCD system, in order to make its diagnoses more accurate, since patients' bodies differ from one another. Of course, such parameter values can be stored in a memory of the WCD system, and the like. Moreover, a programming interface can be made according to embodiments, which receives such measured values of baseline physiological parameters. Such a programming interface may input automatically in the WCD system these, along with other data.

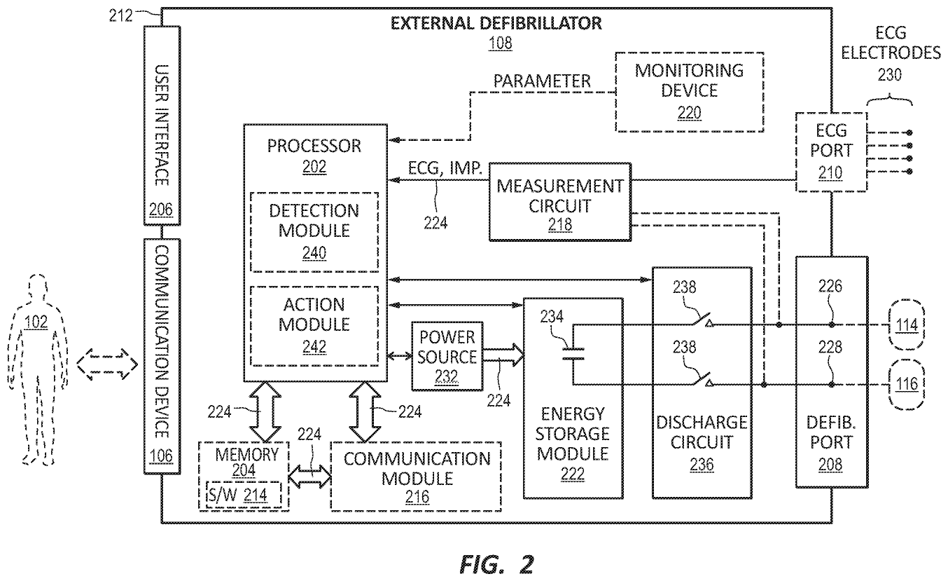

FIG. 2 is a diagram displaying various components of an example external defibrillator 108. The external defibrillator 108 may be an example of the defibrillator 108 described with reference to FIG. 1. The components shown in FIG. 2 may be contained within a single unit or may be separated amongst two or more units in communication with each other. The defibrillator 108 may include a communication device 106, processor 202, memory 204, defibrillation port 208, and ECG port 210, among other components. In some embodiments, the components are contained within a housing 212 or casing. The housing 212 may comprise a hard shell around the components or may comprise a softer shell for increased patient comfort.

The communication device 106, processor 202, memory 204 (including software/firmware code (SW) 214), defibrillation port 208, ECG port 210, communication module 216, measurement circuit 218, monitoring device 220, and energy storage module 222 may communicate, directly or indirectly, with one another via one or more buses 224. The one or more buses 224 may allow data communication between the elements and/or modules of the defibrillator 108.

The memory 204 may include random access memory (RAM), read only memory (ROM), flash RAM, and/or other types. The memory 204 may store computer-readable, computer-executable software/firmware code 214 including instructions that, when executed, cause the processor 202 to perform various functions (e.g., determine shock criteria, determine consciousness of patient, track patient parameters, establish electrode channels, determine noise levels in electrode readings, transfer patient data to a remote storage center, etc.). In some embodiments, the processor 202 may include an intelligent hardware device, e.g., a central processing unit (CPU), a microcontroller, an application-specific integrated circuit (ASIC), etc.

In some embodiments, the memory 204 can contain, among other things, the Basic Input-Output system (BIOS) which may control basic hardware and/or software operations such interactions and workings of the various components of the defibrillator 108, and in some embodiments, components external to the defibrillator 108. For example, the memory 204 may contain various modules to implement the workings of the defibrillator 108 and other aspects of the present disclosure.

In some embodiments, the defibrillator 108 may include a user interface 206. The user interface 206 may be in addition to or part of the communication device 106. The user interface 206 may display an ECG of the patient, a status of the defibrillator 108, a status of a charge (e.g. a battery charge or an energy storage module), and the like.

In some embodiments, the defibrillator 108 may include a defibrillation port 208. The defibrillation port 208 may comprise a socket, opening, or electrical connection in the housing 212. In some instances, the defibrillation port 208 may include two or more nodes 226, 228. The two or more nodes 226, 228 may accept two or more defibrillation electrodes (e.g. defibrillation electrodes 114, 116, FIG. 1). The nodes 226, 228 may provide an electrical connection between the defibrillation electrodes 114, 116 and the defibrillator 108. The defibrillation electrodes 114, 116 may plug into the two or more nodes 226, 228 via one or more leads (e.g. leads 118), or, in some instances, the defibrillation electrodes 114, 116 may be hardwired to the nodes 226, 228. Once an electrical connection is established between the defibrillation port 208 and the electrodes 114, 116, the defibrillator 108 may be able to deliver an electric shock to the patient 102.

In some embodiments, the defibrillator 108 may include an ECG port 210 in the housing 212. The ECG port 210 may accept one or more ECG electrodes 230 or ECG leads. In some instances, the ECG electrodes 230 sense a patient's ECG signal. For example, the ECG electrodes 230 may record electrical activity generated by heart muscle depolarization. The ECG electrodes 230 may utilize 4-leads to 12-leads or multichannel ECG, or the like. The ECG electrodes 230 may connect with the patient's skin.

In some embodiments, the defibrillator 108 may include a measurement circuit 218. The measurement circuit 218 may be in communication with the ECG port 210. For example, the measurement circuit 218 may receive physiological signals from ECG port 210. The measurement circuit 218 may additionally or alternatively receive physiological signals via the defibrillation port 208 when defibrillation electrodes 114, 116 are attached to the patient 102. The measurement circuit 218 may determine a patient's ECG signal from a difference in voltage between the defibrillation electrodes 114, 116.

In some embodiments, the measurement circuit 218 may monitor the electrical connection between the defibrillation electrodes 114, 116 and the skin of the patient 102. For example, the measurement circuit 218 can detect impedance between electrodes 114, 116. The impedance may indicate the effective resistance of an electric circuit. An impedance calculation may determine when the electrodes 114, 116 have a good electrical connection with the patient's body.

In some embodiments, the defibrillator 108 may include an internal monitoring device 220 within the housing 212. The monitoring device 220 may monitor at least one local parameter. Local parameters may include physical state of the patient such as ECG, movement, heartrate, pulse, temperature, and the like. Local parameters may also include a parameter of the WCD system (e.g. WCD 104, FIG. 1), defibrillator 108, environmental parameters, or the like.

In some embodiments, the WCD system 104 may include an internal monitoring device 220 and an external monitoring device (e.g. external monitoring device 124). If both monitoring devices 124, 220 are present, the monitoring devices 124, 220 may work together to parse out specific parameters depending on position, location, and other factors. For example, the external monitoring device 124 may monitor environmental parameters while the internal monitoring device 220 may monitor patient and system parameters.

In some embodiments, the defibrillator 108 may include a power source 232. The power source 232 may comprise a battery or battery pack, which may be rechargeable. In some instances, the power source 232 may comprise a series of different batteries to ensure the defibrillator 108 has power. For example, the power source 232 may include a series of rechargeable batteries as a prime power source and a series of non-rechargeable batteries as a secondary source. If the patient 102 is proximate an AC power source, such as when sitting down, sleeping, or the like, the power source 232 may include an AC override wherein the power source 232 draws power from the AC source.

In some embodiments, the defibrillator 108 may include an energy storage module 222. The energy storage module 222 may store electrical energy in preparation or anticipation of providing a sudden discharge of electrical energy to the patient. In some embodiments, the energy storage module 222 may have its own power source and/or battery pack. In other embodiments, the energy storage module 222 may pull power from the power source 232. In still further embodiments, the energy storage module 222 may include one or more capacitors 234. The one or more capacitors 234 may store an electrical charge, which may be administered to the patient. The processor 202 may be communicatively coupled to the energy storage module 222 to trigger the amount and timing of electrical energy to provide to the defibrillation port 208 and, subsequently, the patient 102.

In some embodiments, the defibrillator 108 may include a discharge circuit 236. The discharge circuit 236 may control the energy stored in the energy storage module 222. For example, the discharge circuit 236 may either electrical couple or decouple the energy storage module 222 to the defibrillation port 208. The discharge circuit 236 may be communicatively coupled to the processor 202 to control when the energy storage module 222 and the defibrillation port 208 should or should not be coupled to either administer or prevent a charge from emitting from the defibrillator 108. In some embodiments, the discharge circuit 236 may include on or more switches 238. In further embodiments, the one or more switches 238 may include an H-bridge.

In some embodiments, the defibrillator 108 may include a communication module 216. The communication module 216 may establish one or more communication links with either local hardware and/or software to the WCD system 104 and defibrillator 108 or to remote hardwire separate from the WCD system 104. In some embodiments, the communication module 216 may include one or more antennas, processors, and the like. The communication module 216 may communicate wirelessly via radio frequency, electromagnetics, local area networks (LAN), wide area networks (WAN), virtual private networks (VPN), RFID, Bluetooth, cellular networks, and the like. The communication module 216 may facilitate communication of data and commands such as patient data, episode information, therapy attempted, CPR performance, system data, environmental data, and so on.

In some embodiments, the processor 202 may execute one or more modules. For example, the processor 202 may execute a detection module 240 and/or an action module 242. The detection module 240 may be a logic device or algorithm to determine if any or a variety of thresholds are exceeded which may require action of the defibrillator 108. For example, the detection module 240 may receive and interpret all of the signals from the ECG port 210, the defibrillation port 208, the monitoring device 220, an external monitoring device, and the like. The detection module 240 may process the information to ensure the patient is still conscious and healthy. If any parameter indicates the patient 102 may be experiencing distress or indicating a cardiac episode, the detection module 240 may activate the action module 242.

The action module 242 may receive data from the detection module 240 and perform a series of actions. For example, an episode may merely be a loss of battery power at the power source 232 or the energy storage module 222, or one or more electrodes (e.g., ECG electrodes, defibrillation electrodes) may have lost connection. In such instances, the action module 242 may trigger an alert to the patient or to an outside source of the present situation. This may include activating an alert module. If an episode is a health risk, such as a cardiac event, the action module 242 may begin a series of steps. This may include issuing a warning to the patient, issuing a warning to a third party, priming the energy storage module 222 for defibrillation, releasing one or more conductive fluids proximate defibrillation electrodes 114, 116, and the like.

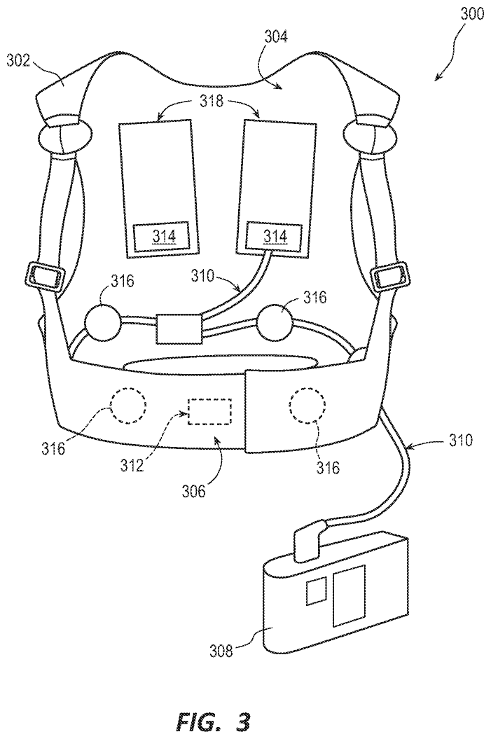

FIG. 3 is a diagram of sample embodiments of components of a WCD system 300 according to exemplary embodiments. The WCD system 300 may be an example of the WCD system 104 describe with reference to FIG. 1. In some embodiments, the WCD system 300 may include a support structure 302 comprising a vest-like wearable garment. In some embodiments, the support structure 302 has a back side 304, and a front side 306 that closes in front of a chest of the patient.

In some embodiments, the WCD system 300 may also include an external defibrillator 308. The external defibrillator 308 may be an example of the defibrillator 108 describe with reference to FIGS. 1 and 2. As illustrated, FIG. 3 does not show any support for the external defibrillator 308, but as discussed, the defibrillator 308 may be carried in a purse, on a belt, by a strap over the shoulder, and the like as discussed previously. One or more wires 310 may connect the external defibrillator 308 to one or more electrodes 312, 314, 316. Of the connected electrodes, electrodes 312, 314 are defibrillation electrodes, and electrodes 316 are ECG sensing electrodes.

The support structure 302 is worn by the patient to maintain electrodes 312, 314, 316 on a body of the patient. For example, the back-defibrillation electrodes 314 are maintained in pockets 318. In some embodiments, the inside of the pockets 318 may comprise loose netting, so that the electrodes 314 can contact the back of the patient. In some instances, a conductive fluid may be deployed to increase connectivity. Additionally, in some embodiments, sensing electrodes 316 are maintained in positions that surround the patient's torso, for sensing ECG signals and/or the impedance of the patient.

In some instances, the ECG signals in a WCD system 300 may comprise too much electrical noise to be useful. To ameliorate the problem, multiple ECG sensing electrodes 316 are provided, for presenting many options to the processor (202. The multiple ECG sensing electrodes 316 provide different vectors for sensing the ECG signal of the patient.

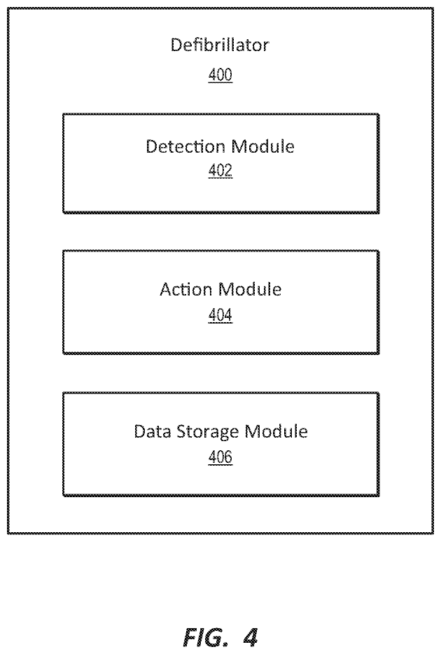

FIG. 4 is a block diagram illustrating components of one example of a defibrillator 400. The defibrillator 400 may be an example of the defibrillator 108 described with reference to FIGS. 1 and 2 and defibrillator 308 described with reference to FIG. 3. In this example, the defibrillator 400 has detection module 402, an alert module 404, and a data storage module 406.

The detection module 402 may be an example of the detection module 240 described with reference to FIG. 2. For example, the detection module may receive and interpret signals received from the ECG port, defibrillation port, an external monitoring device, and the like. The detection module 402 may process all of the data to determine a status of the patient. For example, the detection module 402 may determine if the patient is healthy and conscious. The detection module 402 may also analyze the data for a shockable rhythm or any other irregularities. In some embodiments, the detection module 402 may filter any noise in the signal from the ECG ports and track activity level and other parameters regarding the patient.

The action module 404 may be one example of the action module 242 described with reference to FIG. 2. For example, the action module 404 may receive data from the detection module 402 and perform a series of actions. For example, the detection module 402 may detect an irregular heartbeat or a cardiac event happening and may relay information to the action module 404 which may initiate a series of actions. This may include issuing a warning to the patient, issuing a warning to a third party, priming an energy storage module for defibrillation, releasing one or more conductive fluids proximate defibrillation electrodes, and the like. In other embodiments, the event may be a device event which may indicate one or more issues with the WCD. For example, there may be a loss of battery power or an electrode may have a bad connection.

In some embodiments, the defibrillator 400 may include the data storage module 406. The data storage module 406 may record all of the data and information collected by the defibrillator 400. This may include raw and processed data which may be stored together or separately. In some embodiments, the data may include the signals from the ECG port, defibrillation port, external monitoring device, and the like. For example, the data may include raw heart rate data or ECG signals. In some embodiments, the data may include any analysis or interpretation of the ECG signal such as removing noise from the signal or correlating the signal. The data may also include heart rate over time, heart rate histogram, steps over time, and episodes that a patient may experience. The data may also include device data, including battery life data, performance data, wear data (how often the patient is wearing the WCD), and the like.

In some embodiments, the data storage module 406 may store the information locally, i.e. on system memory 204. In other embodiments, when the data storage module 406 is connected to the internet, the data storage module 406 may upload data to a secure memory. For example, the data storage module 406 may upload information into a remote distributed network hierarchy. This may also be known as blockchain. The remote distributed network may consist of a distributed and immutable ledger allowing the user to track anything, tangible or intangible goods. The blockchain achieves the ledger by generating a chain of blocks with digital timestamp. Each block contains a predetermined amount of data. The blocks link together, and the digital timestamp makes it hard to backdate or tamper with the data.

Once data has been recorded inside the blockchain it becomes very difficult to change it. Each block contains data, a hash of the block, and a hash of the previous block. The data may include raw ECG data, filter ECG data, heart rate trends, activity, shockable events, and the like. The hash is similar to a fingerprint, it identifies the block and the block's contents and the hash is always unique for each block. Once a block is created, the hash is created. If something is changed in the block, the hash will also be changed. Once the hash is changed, it becomes a new block in the chain. The block also stores the hash of the previous block, so each block points to the previous block forming a chain. The first block is the beginning block, or genesis block, and does not store the hash of the previous block as there is none. Once the data is uploaded, it's broadcast and verified on the distributed network. If someone tries to tamper with a block in the chain, permissioned members and validation tools work together to confirm or reject the new data. This process ensures the blockchain remains a safe and secure trusted source.

Prior to uploading the data to the blockchain, the data storage module 406 may divvy and prepare the data for upload. For example, the type of blockchain used may determine the size of the data blocks. Some blockchain protocols may use smaller sized data blocks while others may use larger ones. In still further embodiments, the size of the data block may also depend on the type of data being uploaded and how the data is used. In some embodiments, if the data is all of the data collected and analyzed by the WCD, the data blocks may be smaller. In further embodiments, if the data is event data or other data that is infrequent, the data blocks may be smaller. Additionally, in some embodiments, a patient may have more than one blockchain related to the WCD. This may include compartmentalizing data into separate blocks and then coupling the blocks together in a chain. It may include adding to an already existing chain for the patient or it may include creating a new chain for a patient.

The data storage module 406 may also encrypt the data prior to sending it to the blockchain. Encrypting prior to uploading the data ensures the data is secure. Only authorized parties that download the information may then access it. For example, some blockchain technologies use private and public keys to access information. This is similar to a username and a password. A public key reveals an online identity where a private key can be used to access the information. Multiple levels and forms of encryption may be used to ensure the data is secure and safe and abides by all federal and state regulations.

In some embodiments, the data storage module 406 may maintain a local copy of all of the data. In further embodiments, the data storage module 406 may only store data in a blockchain methodology. In still further embodiments, the data storage module 406 may maintain a secondary storage in a central data storage module.

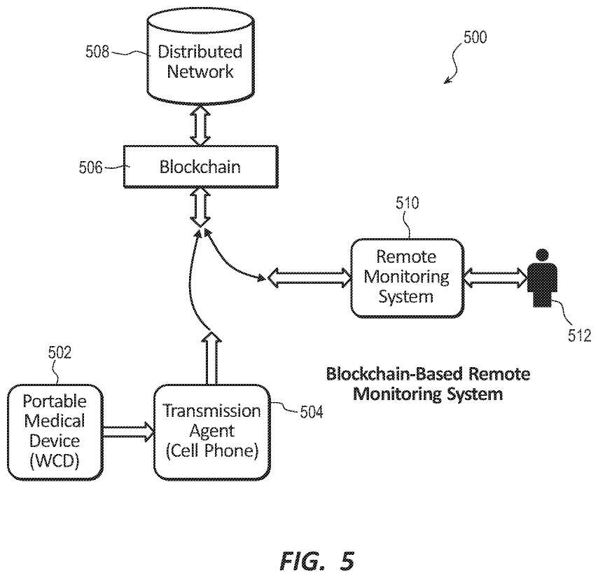

FIG. 5 is a block diagram of an exemplary remote data storage system 500. The system 500 includes a WCD 502, a transmission agent 504, a blockchain storage system 506, a distributed network 508 coupled to the blockchain storage system 506, a remote monitoring system 510, and a system user 512. The WCD 502 may be one example of the WCD 100 described with reference to FIG. 1. The WCD 502 may include a defibrillator such as defibrillator 108, 308, and/or 400.

The WCD 502 may collect and analyze patient and equipment data. The WCD 502 may then encrypt the data and have the transmission agent 504 submit the data to the blockchain storage system 506. The transmission agent may be the defibrillator connected to the internet such as Wi-Fi or may be a secondary device such as a cell phone using cellular protocols.

Once the data is submitted to the blockchain 506, the data may be mined and added to the patient's chain. Mining the data may verify the authenticity of the data to ensure illegitimate or tampered data is not uploaded to the chain. Once the data has been recorded in the blockchain, it is very difficult to change. Each block contains the data, in this case WCD data, a hash (which is like a fingerprint, it identifies a block and all of its contents). When something inside the block is changed, the hash is recalculated. Recalculating the hash generates a new block. The new block also has the hash of the previous block, linking the blocks together in a chain.

The creation of new blocks requires a “proof of work.” In some systems, this takes about ten minutes to complete. If you tamper with one block, you must complete the proof of work for each subsequent block. Because completing the proof of is a lengthy time process, it is difficult to complete prior to a third party noticing the tampering.

Blockchain stores the data on a peer to peer network. When a new block is created, the block is sent to everyone on the network. Each node then verifies the block to make sure it hasn't been tampered with. If the block passes scrutiny, the block is added to the chain. Each node creates consensus. Any tampered block will be rejected by other nodes in the network.

A system user may then wish to access the patient's data stored with blockchain system 506. This may include the patient's doctor, the provider of the WCD, and other medical professionals. The patient may provide access to the system user 512 which may access the blockchain via a remote monitoring system 510. This may enable the patient to grant access to the data without the need to transfer the data, export the data, or modify the data into a different format. With all of the data in recorded immutably, the patient and other parties have the inherent knowledge that the data they are viewing has not been altered. Additionally, multiple parties can access the data at the same time. The original data provider retains control of and access to the patient medical data and can revoke or grant access to various system users 512 as desired.

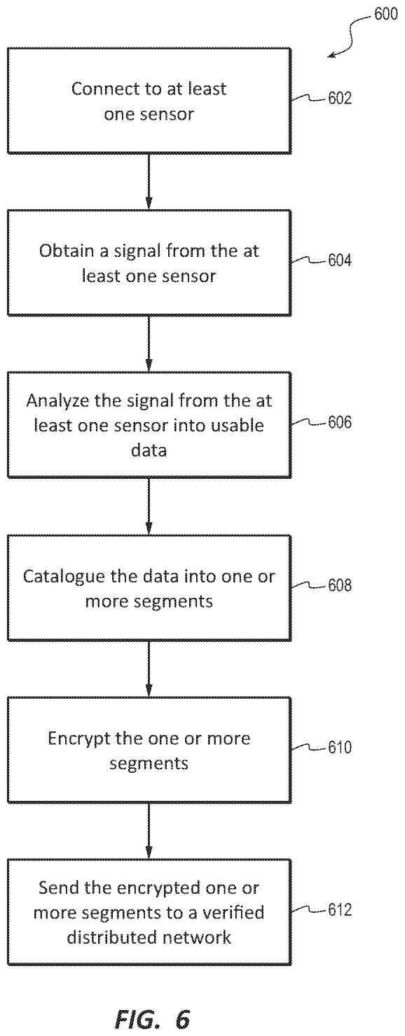

FIG. 6 is a flow chart illustrating an example of a method 600 for WCD systems, in accordance with various aspects of the present disclosure. For clarity, the method 600 is described below with reference to aspects of one or more of the systems described herein.

At block 602, the method 600 may include connecting to at least one sensor. The sensor may include a motion sensor, one or more ECG electrodes, and the like. At block 604, the method 600 may include obtaining a signal from the at least one sensor. For example, the motion sensor may detect a patient movement including steps over time. The ECG electrodes may a patient's ECG signal. In some embodiments, the ECG electrodes may record electrical activity generated by heart muscle depolarization.

At bock 606, the method 600 may analyze the data from the at least one sensor into usable data. In some embodiments, analyzing the signal may include filtering through the raw signal to remove any noise in the system. In further embodiments, the signal may be analyzed to determine a heartrate over time, a heartrate histogram, steps over time, detected ECG arrhythmias, wearability data, heartrate irregularities, and the like. In some embodiments, the data may include episode data consisting of patient medical information regarding a cardiac event that the method 600 detects or chooses to record. It may also include various data such as ECG segments, discrete event records, algorithm decisions and annotations, and the like. In some embodiments, the data may include a trend such as a heart rate trend, a wearability trend, a movement trend, or the like. In further embodiments, the data may include heart rate data, defibrillator status data, wearability data, patient movement data, heartrate over time, heartrate histogram, steps over time, detected electrocardiogram arrhythmias, or the like. In some instances, the data may include a series of time-correlated data that the WCD collects at regular intervals.

At block 608, the method 600 may catalogue the data into one or more segments. For example, the method 600 may be preparing data to be uploaded to a remote distributed network such as the blockchain. Since data is added in small increments to a chain of data, the data needs to be separated into blocks, or segments. The method 600 may divvy the data up by date, duration, time, event, type of data, and in some instances may prepare the raw signal to be uploaded. In some embodiments, the segments may be divided or organized by date, time, duration, cardiac event, or the like. For example, in some embodiments, the method 600 may separate the data into various segments and only send select data to the blockchain. For example, if a physician is viewing the data, the physician may only need to view specific parts of data recorded by the WCD. This may enable the physician to gauge a health of the patient and assess the patient's medical care. Multiple different physicians may be attending to the patient and may need to view this data. Therefore, the blockchain may provide a centralized location for all attending physicians to review the data as well as notes and potentially changes to the patient's medical plan.

In some embodiments, the maker of the WCD system may wish to view the raw data and filtered data to check metrics and improve the workability of the systems. In some embodiments, this may be specific to a patient. In other embodiments, the source of the data (i.e. the patient) may be irrelevant. In this instance, the method 600 may upload the data without any patient identifiers to a chain specifically used by the make of the WCD for device diagnostics.

Once the data is segmented, at block 610, the method 600 may encrypt the segments. Each segment may be separately encrypted. The method 600 may store the encryption key and, in some embodiments, may distribute the encryption key to authorized personnel. Authorized personnel may include doctors, prescribers, health care providers, electronic medical record systems, medical device data aggregation systems, or other types of medical and non-medical data systems. In some embodiments, the method 600 may include encrypting the data using a private key. This private key may be shared with authorized personnel. In some embodiments, the personnel may be provided with various level of access. For example, the access controls may provide client access in various possible ways including all, limited, and no access.

At block 612, the method 600 may include sending the one or more segments to a verified distributed network. The method 600 may transmit the block directly to the internet via the WCD or may use an agent such as a cell phone or tablet. Sending the segments may include uploading the segments to be mined and added to the patient's blockchain. If the patient has yet to establish a blockchain, mining the data may include generating a new blockchain with a genesis block. Once the blockchain is created, or the chain of segmented data, the authorized personnel may access the chain and add additional segments or blocks of notes or annotations to store within the chain. This method of storing data allows multiple health personnel to access and view the data as well as add to the data without changing the original data.

In some embodiments, once data is stored on the blockchain, the method 600 may receive a public and private identifier. The public and private identifier may act similar to a username and password. It may allow a person to recall the data from the blockchain with the username portion and unlock it with the password portion. The method 600 may locally store the public and private identifier locally. This may also be shared to one or more users such as authorized personnel requiring access to the data.

Thus, the method 600 may provide for storing WCD data. It should be noted that the method 600 is just one implementation and that the operations of the method 600 may be rearranged or otherwise modified such that other implementations are possible.

A person skilled in the art will be able to practice the present invention after careful review of this description, which is to be taken as a whole. Details have been included to provide a thorough understanding. In other instances, well-known aspects have not been described, in order to not obscure unnecessarily this description.

Some technologies or techniques described in this document may be known. Even then, however, it is not known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

What is claimed is:

1. A method to store data collected by a wearable cardioverter defibrillator (WCD), the method comprising:
connecting to at least one sensor;
obtaining a signal from the at least one sensor;
analyzing the signal from the at least one sensor into usable data;
cataloguing the data into one or more segments;
encrypting the one or more segments; and
sending the encrypted one or more segments to a verified distributed network.

2. The method of claim 1, further comprising:
storing the encryption key; and
distributing the encryption key to authorized personnel.

3. The method of claim 1, further comprising:
receiving a public and private identifier for the verified distributed network;
locally storing the public and private identifier; and
distributing the public and private identifier to authorized personnel.

4. The method of claim 1, wherein sending the encrypted one or more segments includes initiating a new data chain in the verified distributed network.

5. The method of claim 4, further comprising:
initiating segment mining to the remote distributed network.

6. The method of claim 1, wherein the data includes one of heart rate data, defibrillator status data, wearability data, patient movement data, heartrate over time, heartrate histogram, steps over time, detected electrocardiogram arrhythmias, and some combination thereof.

7. The method of claim 1, wherein the segments are organized by at least one of a date, time, duration, cardiac event, or some combination thereof.

8. A wearable cardiac defibrillator (WCD) system for monitoring health of a patient wearing the WCD system, the system comprising:
at least one sensor positioned to gather data about the patient
one or more memories, the one or more memories configured to store patient data; and
one or more processors configured to cause the system to:
receive a signal from the at least one sensor;
analyze the signal from the at least one sensor into usable data;
catalogue the usable data into one or more segments;
encrypt the one or more segments;
send the one or more encrypted segments to a remote distributed network.

9. The WCD system of claim 8, wherein the processor is further configured to:
store the encryption key; and
distribute the encryption key to authorized personnel.

10. The WCD system of claim 8, wherein the processor is further configured to:
receive a public and private identifier for the verified distributed network;
locally store the public and private identifier; and
distribute the public and private identifier to authorized personnel.

11. The WCD system of claim 8, wherein sending the encrypted one or more segments includes initiating a new data chain in the verified distributed network.

12. The WCD system of claim 11, wherein the processor is further configured to:
initiate segment mining to the remote distributed network.

13. The WCD system of claim 8, wherein the data includes one of heart rate data, defibrillator status data, wearability data, patient movement data, heartrate over time, heartrate histogram, steps over time, detected electrocardiogram arrhythmias, and some combination thereof.

14. The WCD system of claim 17, wherein the segments are catalogued by at least one of a date, time, duration, cardiac event, or some combination thereof.

15. A method to store patient in a remote distributed network, the method comprising:
positioning at least one electrocardiogram (ECG) sensing electrodes to measure electrical activity of a heart of a person;
receiving at least one ECG signals from the at least one ECG electrodes;
analyzing the signal into usable data including at least one of a heartrate over time, heartrate histogram, and detected ECG arrhythmias;
cataloguing the usable data into one or more segments based at least in part on a size of the data;
encrypting the one or more segments;
sending the one or more encrypted segments to a remote distributed network.
